(19) United States
(12) Patent Application Publication
(10) Pub. No.: US 2023/0092634 A1
(43) Pub. Date: Mar. 23, 2023

(54) SYSTEM AND APPARATUS FOR NANOPORE SINGLE MOLECULE SEQUENCING

(71) Applicants: MGI Holdings Co., Limited, Shenzhen (CN); BGI Shenzhen, Shenzhen (CN)

(72) Inventors: Shifeng Li, Fremont, CA (US); Hemanth Kanekal, San Jose, CA (US); Yuning Zhang, Shenzhen (CN); Quanxin Yun, Shenzhen (CN)

(21) Appl. No.: 17/922,656

(22) PCT Filed: Apr. 27, 2021
(86) PCT No.: PCT/CN2021/090243

§ 371 Date: Nov. 1, 2022

(30) Foreign Application Priority Data
May 7, 2020 (CN) PCT/CN2020/088904

(51) Int. Cl. G01N 33/487

(52) U.S. Cl. CPC G01N 33/48721; H03M 1/122

(57) ABSTRACT

An integrated circuit for controlling a sensor chip capable of sensing various materials includes a plurality of amplifier clusters, a plurality of analog multiplexers, and at least one analog-to-digital converter coupled the analog multiplexers and configured to generate digital code values representative of electrical signals. Each of the amplifier clusters include four amplifiers, each amplifier has a first input coupled to a sensor of the sensor chip, and a second input coupled to a programmable voltage reference. Each one of the analog multiplexers is coupled to one of the amplifier clusters and configured to selectively pass through an electrical signal to the at least one analog-to-digital converter.

### FIELD

The present invention relates generally to an analysis instrument, and more particularly to systems and integrated devices for high-throughput biochemical analysis and methods of operating the same.

### BACKGROUND OF THE INVENTION

High-throughput analysis of chemical and/or biochemical species is an important tool in the field of diagnostics and therapeutics. Sensor chips can be designed to detect specific sequences, analyze gene expression patterns, identify specific allelic variations, determine copy number of DNA sequences, and identify, on a genome-wide basis, binding sites for proteins (e.g., transcription factors and other regulatory molecules). In a specific example, the advent of the human genome project required that improved methods for sequencing nucleic acids, such as DNA (deoxyribonucleic acid) and RNA (ribonucleic acid), be developed. Determination of the entire 3,000,000,000 base sequence of the haploid human genome has provided a foundation for identifying the genetic basis of numerous diseases.

High-throughput analyses, such as massively parallel DNA sequencing, often utilize flow cells, which contain arrays of chemicals and/or biological species available for analysis. Flow cells are often made with a microfluidic housing integrated with a biological chip, for example, a silicon-based sensor chip, to form a microfluidic apparatus.

Single molecule characterization using nanopore membranes requires detection and measurement of very low ionic current flowing through the nanopores. The magnitude of the ionic current through a nanopore is on the order of a few tens or hundreds of picoamps (pA). It is very challenging to detect any changes in such low-level current through a nanopore.

### BRIEF SUMMARY OF THE INVENTION

Embodiments of the present invention provide an integrated circuit for controlling a sensor chip capable of sensing various materials. The integrated circuit includes a plurality of amplifier clusters, a plurality of analog multiplexers, and at least one analog-to-digital converter coupled the analog multiplexers and configured to generate digital code values representative of electrical signals. Each of the amplifier clusters include four amplifiers, each amplifier has a first input coupled to a sensor of the sensor chip, and a second input coupled to a programmable voltage reference. Each one of the analog multiplexers is coupled to one of the amplifier clusters and configured to selectively pass through an electrical signal to the at least one analog-to-digital converter.

In one embodiment, the integrated circuit further includes a digital-to-analog converter configured to generate the programmable voltage reference in response to a digital input signal.

In one embodiment, each amplifier cluster includes four amplifiers, and each analog multiplexer includes four inputs, each input being coupled to an output of an amplifier in the amplifier cluster.

In one embodiment, the integrated circuit also includes a correlated double sampling and low-pass filtering circuit coupled to the plurality of analog multiplexers and configured to reduce noise and offset voltage and drift of the integrated circuit.

In one embodiment, the integrated circuit also includes a timing and control circuit configured to provides control signals to the plurality of amplifier clusters, the plurality of first analog multiplexers, and the at least one ADC.

In one embodiment, the plurality of amplifier clusters, the plurality of first analog multiplexers, and the at least one ADC are supplied by individual voltage supplies that are physically and electrically separated from each other.

In one embodiment, the integrated circuit also includes a self-calibration and test circuit configured to calibrate the plurality of amplifier clusters and analyze a plurality of data flows from the amplifier clusters to the at least one ADC.

In one embodiment, the integrated circuit also includes a plurality of second analog multiplexers arranged between the plurality of first analog multiplexers and the at least one ADC and configured to sequentially provide the selectively pass through electrical signals to the at least one ADC.

According to an embodiment, a nanopore flow cell system is provided. The nanopore flow cell system includes a sensor chip having a plurality of sensors, an integrated circuit (IC) configured to receive an electrical signal of a sensor of the sensor chip and output a digital code value representative of the electrical signal, and an interface device coupled to the integrated circuit and configured to process the digital code value received from the IC and provide control signals to the IC according to the processed digital code value. The IC includes a plurality of amplifier clusters, each of the amplifier clusters comprising a number of amplifiers, each amplifier having a first input coupled to a sensor of the sensor chip and a second input coupled to a programmable voltage reference, a plurality of analog multiplexers, each one of the analog multiplexers being coupled to one of the amplifier clusters and configured to selectively pass through an electrical signal, and at least one analog-to-digital converter (ADC) coupled the analog multiplexers and configured to generate digital code values representative of electrical signals.

In one embodiment, the integrated circuit is a complementary metal oxide semiconductor (CMOS) application specific integrated circuit (ASIC), and the interface device is a field programmable gate array (FPGA).

In one embodiment, the nanopore flow cell system further includes a substrate disposed between the integrated circuit and the sensor chip. The integrated circuit is in communication with the sensor chip through a plurality of through-silicon vias extending to the substrate.

In one embodiment, the integrated circuit is in communication with the interface device via a four-wire serial peripheral interface (SPI) and a low-voltage differential signaling (LVDS) port.

In one embodiment, the programmable voltage reference is configured to provide a bias voltage to the sensor chip for one of DNA strain unblocking, nanopore membrane characterization, or protein insertion.

In one embodiment, the interface device is configured to perform arithmetic operations on the digital code values received from the IC and transmits control signals to the IC according to results of the arithmetic operations.

In one embodiment, the sensor chip is disposed on a sensor substrate, the integrated circuit is disposed on a second substrate, the sensor chip and the integrated circuit are coupled together through a set of pogo pins.

Embodiments of the present invention also provide a method of operating an integrated circuit (IC) configured to control a sensor chip. The method may include receiving an electrical signal by the IC from the sensor chip, amplifying the received electrical signal using an amplifier having a first input coupled to the sensor chip and a second input coupled to a programmable voltage reference, converting the amplified electrical signal to a digital code value representative of the amplified electrical signal by an analog-to-digital converter (ADC), outputting the digital code value to an interface device, and varying the programmable voltage reference in response to control signals received by the interface device.

In one embodiment, the method further includes multiplexing the amplified electrical signal through multiple stages of analog multiplexers prior to converting the amplified electrical signal to the digital code value.

Embodiments of the present invention also provide a method of operating a flow cell analysis system. The method includes providing a sensor chip including an array of sensors, providing an integrated circuit including a plurality of amplifiers, each amplifier configured to scale an electrical signal of one of the sensors, selectively passing through a portion of the scaled electrical signals, converting the portion of the scaled electrical signals to digital codes representative of the portion of the scaled electrical signals, outputting the digital codes to an external device using a high-speed serial interface, analyzing the digital codes by the external device to obtain an analysis result, and applying a programmable voltage reference to the sensor chip in response to the analysis result.

Numerous benefits are achieved by way of the present invention over known techniques. For example, embodiments of the present invention provide systems, devices and methods that utilize a flow cell including a sensor chip, a CMOS application specific integrated circuit (ASIC), and a field programmable gate array (FPGA). The flow cell is mounted on a first substrate, and the CMOS ASIC and the FPGA are mounted on a second substrate. The flow cell can be connected to the CMOS ASIC chip via a set of pogo pins when the first and second substrates are brought together. The flow cell is disposable whereas the CMOS ASIC and the FPGA are reusable. These and t=other embodiments of the invention along with many of its advantages and features are described in more detail in conjunction with the text below and attached figured.

### BRIEF DESCRIPTION OF THE DRAWINGS

The accompanying drawings form a part of the present disclosure, that described exemplary embodiments of the present invention. The drawings together with the specification will explain the principled of the invention.

FIG. 1A is a simplified block diagram illustrating a nanopore analysis system according to an embodiment of the present disclosure.

FIG. 1B is a simplified cross-sectional view diagram illustrating a nanopore flow cell system according to an embodiment of the present disclosure.

FIG. 1C is a simplified cross-sectional view diagram illustrating a nanopore flow cell apparatus according to another embodiment of the present disclosure.

FIG. 2A is a top plan view of a sensor chip according to another embodiment of the present disclosure.

FIG. 2B is an enlarged portion of the sensor chip of FIG. 2A.

FIG. 2C is a simplified cross-sectional view illustrating a portion of the sensor chip including a well and an electrode according to an embodiment of the present disclosure.

FIG. 3A is a flow chart illustrating a method 300A for preparing a nanopore flow cell system according to an embodiment of the present disclosure.

FIG. 3B is a graph of an electric current illustrating a condition where a nanopore is blocked and then unblocked according to an embodiment of the present disclosure.

FIG. 3C is a simplified block diagram illustrating the blocking condition of a molecule strand in a nanopore when the ASIC applies a bias voltage V to draw the molecule strand to the nanopore and block the nanopore corresponding to the state B of FIG. 3B.

FIG. 3D is a simplified block diagram illustrating that the ASIC applies a reverse bias voltage to unblock the nanopore corresponding to the state C of FIG. 3B.

FIG. 4 is a simplified block diagram illustrating a multiple-sensor integrated chip architecture for a nanopore flow cell system according to various embodiments of the present disclosure.

FIG. 5 is a schematic block diagram illustrating a two-stage amplifier 50 according an embodiment of the present disclosure.

FIG. 6 is a timing diagram illustrating an exemplary readout cycle of a nanopore based on a correlated double sampling process according to an embodiment of the present disclosure.

FIG. 7 is a simplified flow chart illustrating a DNA unblocking process according to an embodiment of the present disclosure.

FIG. 8 is a simplified flow chart illustrating a membrane characterization process according to an embodiment of the present disclosure.

FIG. 9 is a simplified flow chart illustrating a protein insertion process according to an embodiment of the present disclosure.

FIG. 10 is a simplified flow chart illustrating a short-circuit prevention process according to an embodiment of the present disclosure.

FIG. 11 is a simplified flow chart illustrating a method of operating a flow cell analysis system according to an embodiment of the present disclosure.

FIG. 12 is a simplified flow chart illustrating a method of operating an integrated circuit which is configured to control a sensor chip having a plurality of sensor elements according to an embodiment of the present disclosure.

### DETAILED DESCRIPTION OF THE INVENTION

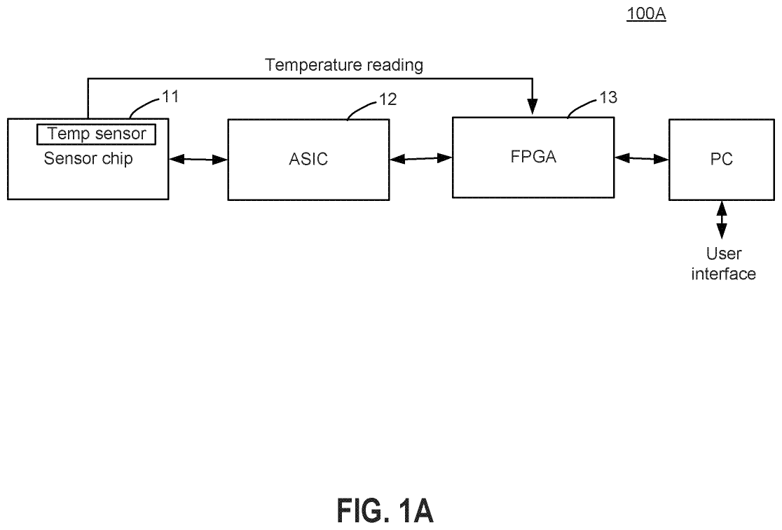

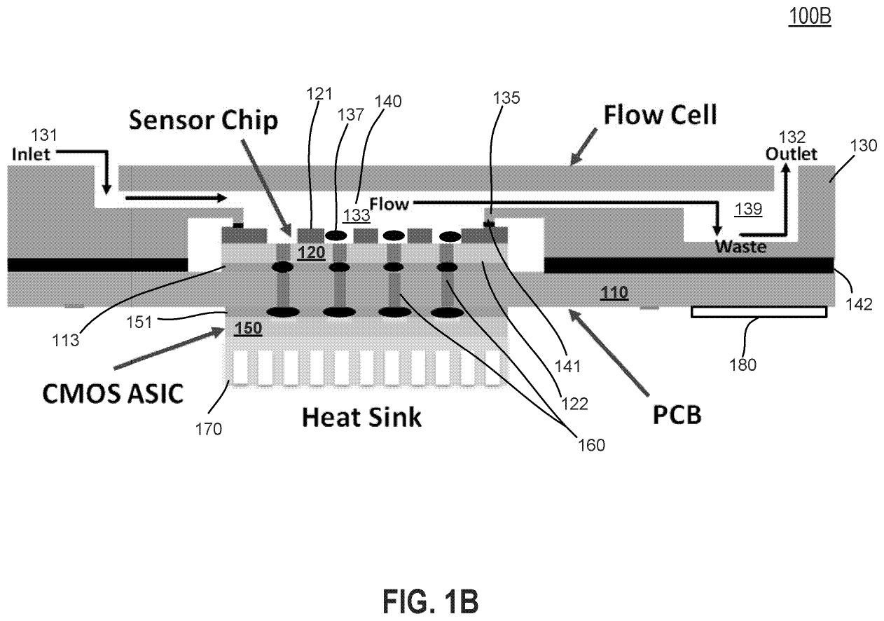

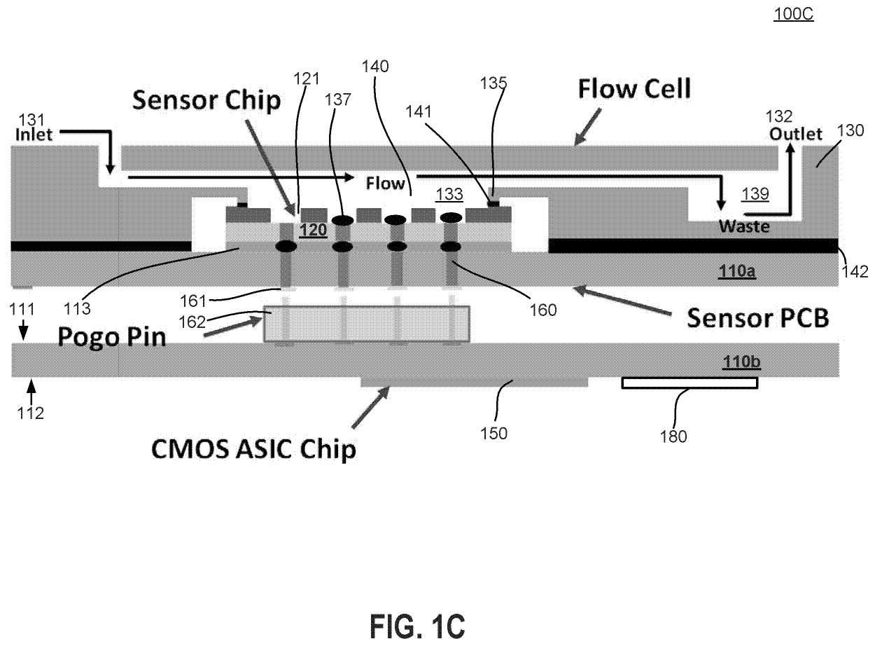

FIG. 1A is a simplified block diagram illustrating a nanopore flow cell analysis system 100A according to an embodiment of the present disclosure. The nanopore flow cell analysis system 100A includes, but not limited to, a sensor chip 11, an ASIC (application specific integrated circuit) chip 12, and an FPGA (field programmable gate array) 13. The sensor chip 11, the ASIC 12, and the FPGA 13 are communicatively coupled to each other so that the sensor chip 11 can be prepared to measure data with respect to a target sample (e.g., a biological or biochemical sample) using nanopores and sensor elements integrated therein. The FPGA 13 is further in communication with a computing device PC (e.g., a personal computer, a PDA, a laptop, and the like) for storing the measured data and for providing a user interface. The nanopore flow cell analysis system 100A can prepare or control the operating conditions of the sensor chip 11 and the ASIC 12, e.g., perform DNA sequencing, characteristics measurement and control of the membrane, protein insertion, control the flow of fluids into and out of the sensor chip 11, unblock blocked nanopores, perform “house-keeping functions,” such as constantly checking for short circuit and other fail conditions, reading junction temperature of the sensor chip, channel shut-off, and self-calibration or user-initiated test via the PC.

In one embodiment, the sensor chip 11 includes an array of sensor elements disposed in an array of nanopores, each sensor element is configured to output an electrical signal in response to a physical condition of a nanopore. In one embodiment, the sensor chip 11 may include a plurality of field effect transistors (FETs), each of the FETs can output an electrical signal indicating a change to an impedance or a characteristic of the FET according to a biochemical material. The ASIC 12 includes a plurality of amplifiers, each amplifier is configured to amplify an electrical signal provided by one of the sensor elements, and a plurality of multiplexers configured to selectively pass through an amplified electrical signal of a sensor element to a number of signal paths, where the number of signal paths is smaller than the number sensor elements or the number of amplifiers. The ASIC 12 also includes one or more analog-to-digital converters configured to receive the amplified electrical signals on the signal paths and convert the selected amplified electrical signals to digital codes or digital words representative of the selected amplified electrical signals, and a controller configured to control the multiplexers to select or pass through electrical signals of the sensor elements that have acceptable performance to the number of signal paths. The ASIC 12 further include a high-speed link configured to output the digital codes to the FPGA 13 and an interface port configured to receive control data and instructions from the FPGA 13. The FPGA 13 receives the digital codes from the ASIC 12, processes the digital codes and provides instructions to the ASIC 12 based on the received digital codes. The FPGA 13 may also provide results of the processed digital codes to the PC for analysis and display. The FPGA 13 may also receive temperature information from a temperature sensor embedded in the sensor chip 11 and provides one or more feedback signals to the sensor chip through the ASIC 12. The nanopore flow cell analysis system 100A can operate autonomously and/or in an interactive mode with a user via a PC. These and other features of the nanopore flow cell analysis system 100A will be described further in detail below.

FIG. 1B is a simplified cross-sectional view diagram illustrating a nanopore flow cell (NFC) apparatus 100B according to an embodiment of the present disclosure. Referring to FIG. 1B, the NFC apparatus 100B includes a substrate 110, a sensor chip 120 attached to the substrate 110 with an attach adhesive layer 113, and a microfluidic housing 130 overlying the sensor chip 120 and the PCB 110. The microfluidic housing 130 is attached to the sensor chip 120 using a first adhesive layer 141 to form a flow cell, and the microfluidic housing is attached to the PCB 110 using a second adhesive layer 142 to provide mechanical support. The sensor chip 120 has a front surface (also referred to as a top surface) 121 and a back (or bottom) surface 122. In one embodiment, the substrate 110 may be a printed circuit board (PCB), and the sensor chip 120 may be capable of biological analysis. It will be appreciated that the substrate 110 described herein is not limited to the PCB, and other substrate can also be used, for example, semiconductor (e. g., silicon) substrate, glass substrate, ceramic substrate, etc.

The microfluidic housing 130 has an inlet 131, an outlet 132, and a first cavity 133. The microfluidic housing 130 can have an inner sidewall 135 adjacent to the cavity 133. The inner sidewall 135 is attached to the sensor chip 120 using the first adhesive layer 141 to form a flow cell 140 with a hermetic seal. As used herein, a hermetic seal refers to a sealing that is airtight and liquid tight, which excludes the passage of air, gases, and liquids. The flow cell 140 includes a channel formed by the cavity 133 between the microfluidic housing 130, inner sidewalls 135 of the microfluidic housing, and the sensor chip 120. As an example application, a biological sample 137 can be introduced through the inlet 131 of the flow cell 140 into the cavity 133, where the sensor elements (also referred to as sensors) in the sensor chip 120 can determine the properties of the biological sample 137. Afterwards, the biological sample 137 can be transferred from the cavity 133 to a waste reservoir 139, and then removed therefrom through the outlet 132 of the flow cell 140.

The first adhesive layer 141 forms the hermetic seal between the microfluidic housing 130 and the sensor chip 120 that is air tight and liquid tight. Further, the first adhesive layer 141 is compatible with the materials used in the flow cell. On the other hand, the second adhesive layer 142 is configured to provide mechanical strength in the joint between the microfluidic housing 130 and the PCB 110. In some examples, the second adhesive layer 142 is thicker than the first adhesive layer 141. A distance 144 between a bottom surface of the outer sidewall 136 of the microfluidic housing and the PCB 110 is greater than the distance 145 between a bottom surface of the inner sidewall 135 of the microfluidic housing and the sensor chip 120.

In some embodiments, for bonding microfluidic housing 130 to the sensor chip 120 and PCB 110, the first and second adhesive layers are first formed, and then the microfluidic housing 130 is picked up and disposed to contact the sensor chip 120 and PCB 110. In some embodiments, the NFC apparatus 100B is designed in a way that the first adhesive layer can be in a solid form and can have a well-defined thickness. On the other hand, the second adhesive layer is sufficiently thick and is in liquid form before curing so that the second adhesive layer's bond line thickness is self-adjustable. In other words, it can fill the space required by the structure of the microfluidic apparatus, which can be influenced by the first adhesive layer thickness, sensor thickness, die attach glue thickness, PCB surface unevenness, and the step of wire bond cavity in the microfluidic apparatus, etc. Here, the bond line thickness refers to the thickness of an adhesive layer between the bottom surface of the device structure above the adhesive layer and the top surface of the device structure below the adhesive layer. Depending on the context, the term “bond line thickness” can refer to a pre-cure bond line thickness or a post-cure bond line thickness of an adhesive layer.

Referring still to FIG. 1B, the NFC apparatus 100B further includes a CMOS application specific integrated circuit (ASIC) device 150 attached to the back side (the side opposite of the front side to which the sensor chip 120 is attached) of PCB 110. In one embodiment, the CMOS ASIC device 150 is attached to the PCB 110 with a second adhesive layer 151. The CMOS ASIC device 150 is in communication with sensor chip 120 through a plurality of through-silicon vias (TSV) 160. In one embodiment, a heat sink 170 is attached to the CMOS ASIC device 150 by a clip or an adhesive (glue) for heat dissipation. In one embodiment, a FPGA 180 may be assembled to or mounted on the PCB 110 and is in communication with the ASIC device 150 through metal wirings on and within the substrate (PCB) 110.

FIG. 1C is a simplified cross-sectional view diagram illustrating a nanopore flow cell (NFC) apparatus 100C according to an embodiment of the present disclosure. Referring to FIG. 1C, the NFC apparatus 100C includes a sensor substrate 110a, a sensor chip 120 attached to the sensor substrate 110a with an attach adhesive layer 113, and a microfluidic housing 130 overlying the biological chip 120 and the sensor substrate 110a. The sensor chip 120 includes a plurality of sensors (sensor elements) arranged in an array of nanopores, each of the sensors is configured to produce an electrical signal in response to a condition of the nanopore. The microfluidic housing 130 is attached to the biological chip 120 using a first adhesive layer 141 to form a flow cell, and the microfluidic housing is attached to the sensor substrate 110a using a second adhesive layer 142 to provide mechanical support. In one embodiment, the sensor substrate 110a may be a printed circuit board (PCB), and the sensor chip 120 may be capable of biological analysis. It will be appreciated that the substrate described herein is not limited to the PCB, and other substrate can also be used, for example, semiconductor (e. g., silicon) substrate, glass substrate, ceramic substrate, etc. The microfluidic housing 130 has an inlet 131, an outlet 132, and a first cavity 133. The microfluidic housing 130 can have an inner sidewall 135 adjacent to the cavity 133, and the inner sidewall 135 is attached to the biological chip 120 using the first adhesive layer 141 to form a flow cell 140 with a hermetic seal. The flow cell 140 includes a channel formed by the cavity 133 between the microfluidic housing 130, inner sidewalls 135 of the microfluidic housing, and the biological chip 120. The flow cell 140 also has the inlet 131 and the outlet 132. A biological sample 137 can be introduced through the inlet 131 into the cavity 133, where the sensor elements (also referred to as sensors) in the sensor chip 120 can determine the properties of the biological sample 137. Afterwards, the biological sample 137 can be transferred from the cavity 133 to a waste reservoir 139, and then removed therefrom through the outlet 132. The flow cell structure of the NFC apparatus 100C is similar to the structure of the NFC apparatus 100B and will not be further described herein for the sake of brevity.

The NFC apparatus 100C further includes a plurality of pads 161 disposed on a second surface (or bottom surface) of the sensor substrate 110a opposite the first surface (or top surface) having the sensor chip 120 mounted thereon. The pads 161 are connected to the sensors of the sensor chip 120 through a plurality of through-silicon vias (TSV) 160.

The NFC apparatus 100C further a second substrate 110b having a first surface 111 and a second surface 112 opposite the first surface, a set of pogo pins 162 mounted on the first surface 111 and configured to contact the pads 161 when the sensor substrate 110a and the second substrate 110b are brought together. The NFC apparatus 100C also includes a CMOS ASIC chip 150 mounted on the second surface 112 of the second substrate 110b. The NFC apparatus 100C also includes an FPGA 180 mounted on the second surface 112 of the second substrate 110b and in communication with the CMOS ASIC chip 150. In one embodiment, the flow cell mounted on the sensor substrate 110a is a single-use flow cell, so that it is disposable, whereas the second substrate 110b having the CMOS ASIC chip and the FPGA mounted thereon is reusable.

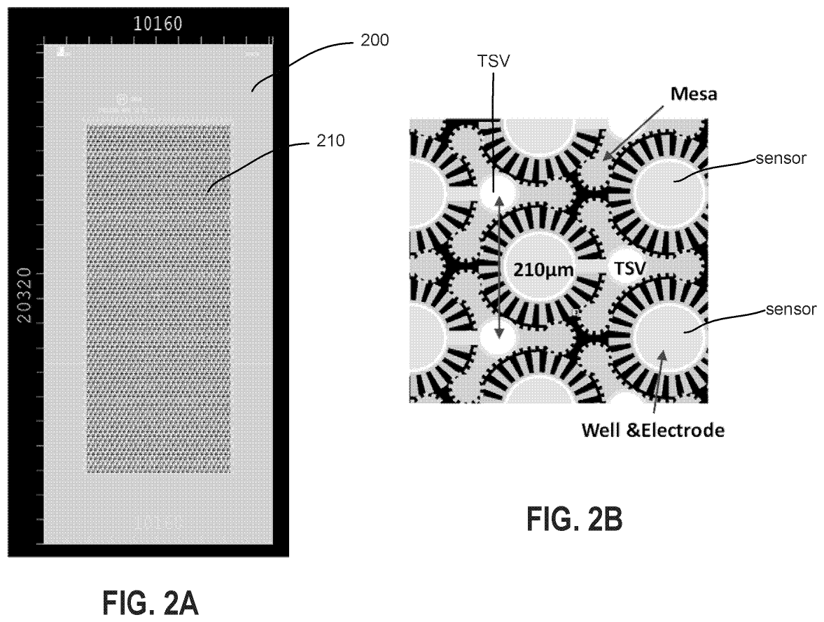

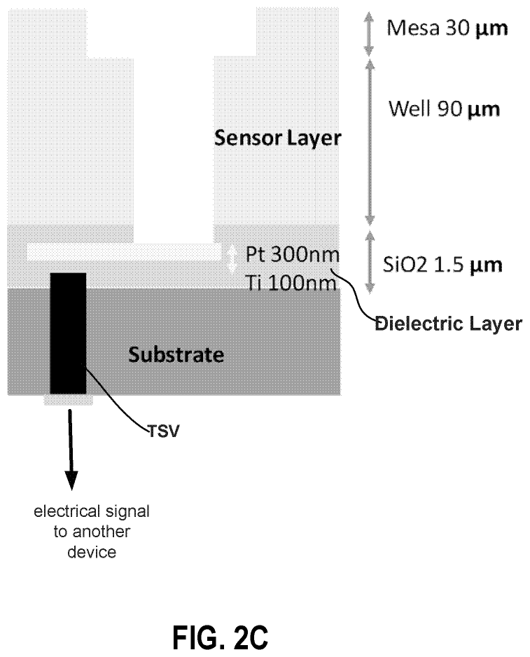

FIG. 2A is a top plan view illustrating a sensor chip 200 according to an embodiment of the present disclosure. The sensor chip 200 may include an array of sensors 210 to allow a simultaneous measurement of a plurality of samples (biological or biochemical material) disposed on its surface. The number of available sensors depends on the sensor density and the dimension of the array or the sensor chip. It is noted that although one sensor chip is shown, one of skill in the art will appreciate that multiple sensor chips can be used in parallel so that the overall throughput can be significantly increased or the readout rate can be reduced to reduce noise. In one embodiment, a plurality of sensors are arranged on a substrate, each sensor of the plurality of sensors has a circular sample well configured to retain a biological sample. The plurality of sensors are separated from each other by a mesa structure.

FIG. 2B is an enlarged portion of the sensor chip of FIG. 2A. Referring to FIG. 2B, a sensor including a well and an electrode provides an electrical signal through a through-silicon via (TSV) to a CMOS ASIC chip. In one embodiment, the TSV has a diameter of equal to or less than 50 μm and a pitch between two TSVs is about 210 μm.

FIG. 2C is a simplified cross-sectional view illustrating a portion of the sensor chip including a well and an electrode according to an embodiment of the present disclosure. Referring to FIG. 2C, a sensor has a sensor layer and a mesa structure on an upper surface of the sensor layer and surrounding the sensor layer. A dielectric is disposed between the substrate and the sensor layer. The dielectric layer may include silicon dioxide (SiO₂). A stack of metal layers is disposed at the bottom of the sensor well and within the dielectric layer and is configured to provide an electrical signal outputted by the sensor to another device (e.g., an ASIC) through the through-silicon via (TSV). In one embodiment, the well has a thickness of about 90 μm, the mesa structure surrounding the well has a thickness of about 30 μm, and the dielectric layer has a thickness of about 1.5 μm. In one embodiment, the stack of metal layers may include a platinum (Pt) layer having a thickness of 300 nm and a titanium (Ti) layer having a thickness of 100 nm.

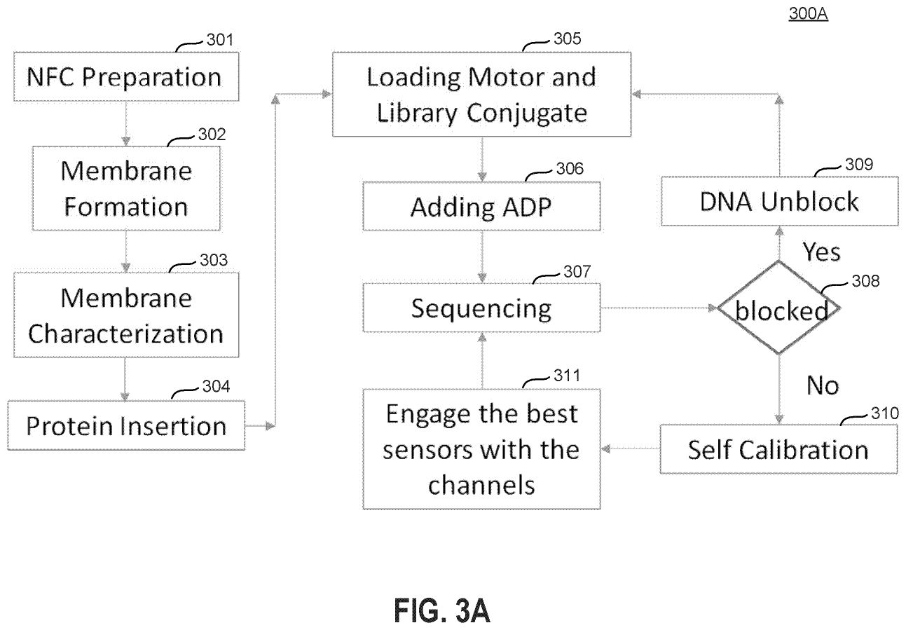

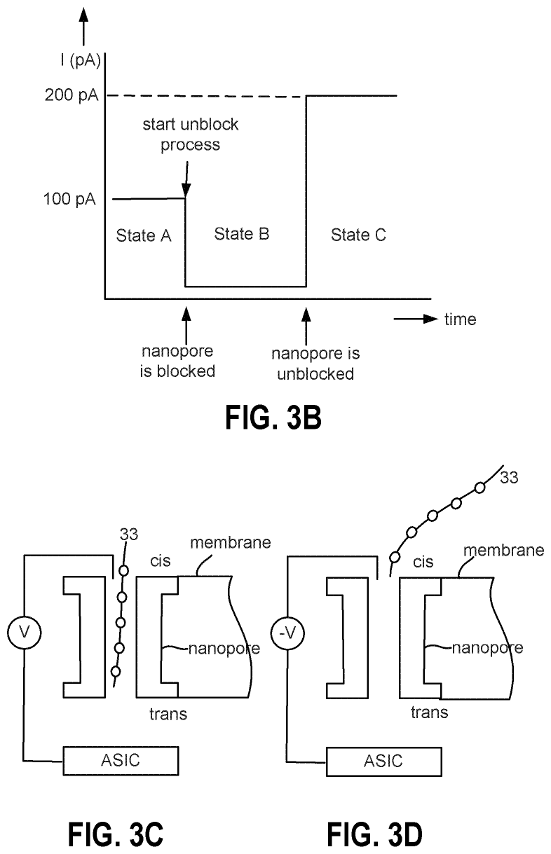

FIG. 3A is a flow chart illustrating a method 300A for preparing a nanopore flow cell system according to an embodiment of the present disclosure. Referring to FIG. 3A, at block 301, the method begins with a nanopore flow cell (NFC) preparation, e.g., by providing a nanopore flow cell analysis system 100A as shown in FIG. 1A. For example, the system 100A may include the sensor chip 11, the ASIC 12, and the FPGA 13. A membrane is formed on the sensor chip at block 302. At 303, the membrane is characterized. The membrane characterization may include verifying whether the membrane is in good working condition by sending a current through the membrane. When the current is below a predetermined value, the membrane is determined to be in good working condition. The membrane is determined to be broken when the current exceeds the predetermined value. At block 304, a voltage is applied to the membrane to deform the membrane for protein (nanopore) insertion. At block 305, motor molecules and library conjugate are loading to the protein (nanopore). At block 306, ADP is added to drive the motor molecules. At block 307, the sequence of the motor molecules is determined as the motor molecules pass through the protein (nanopore). The sequencing may be based on an electric current change as a motor molecule passes through a nanopore. The method determines whether the electric current is over a predetermined current level at block 308. If the electric current is below the predetermined current level (Yes at block 308), the method determines that the nanopore is blocked and causes the ASIC to apply a reverse voltage to the blocked nanopore in order to unblock the nanopore, i.e., push the molecule (DNA) out of the blocked nanopore. When the nanopore is unblocked at block 309, the method repeats blocks 305, 306, 307, and 308 for molecule (DNA) sequencing. When the method determines that the nanopore is not blocked (No at block 308), the method proceeds to perform a self-calibration at block 310, then engage the best sensors with the channels for DNA sequencing at block 311.

FIG. 3B is a graph of an electric current illustrating a condition where a nanopore is blocked and then unblocked according to an embodiment of the present disclosure. As shown in FIG. 3B, the x-axis represents time and the y-axis represents the electric current I in pA, the electric current may be in one of the three states: state A, state B, and state C. State A shows an example of the electric current when the molecule passes through an unblocked nanopore. In the example shown, the electric current is about 100 pA when the molecule passes through the nanopore, which is unblocked. State B shows an example of the electric current when the nanopore is blocked by the molecule. In the example shown, the electric current is significantly below 100 pA. When this condition is detected, the method causes the ASIC chip to reverse the voltage applied to the nanopore to push the molecule out of the blocked nanopore. Once the nanopore is unblocked, the electric current may increase to a high current level, e.g., 200 pA, indicating that the unblocked state (state C) of the nanopore.

FIG. 3C is a simplified block diagram illustrating the blocking condition of a molecule strand 33 in a nanopore when the ASIC applies a bias voltage V to draw the molecule strand 33 to the nanopore and block the nanopore corresponding to the state B in FIG. 3B. FIG. 3D is a simplified block diagram illustrating that the ASIC applies a reverse bias voltage −V to unblock the nanopore corresponding to the state C in FIG. 3B.

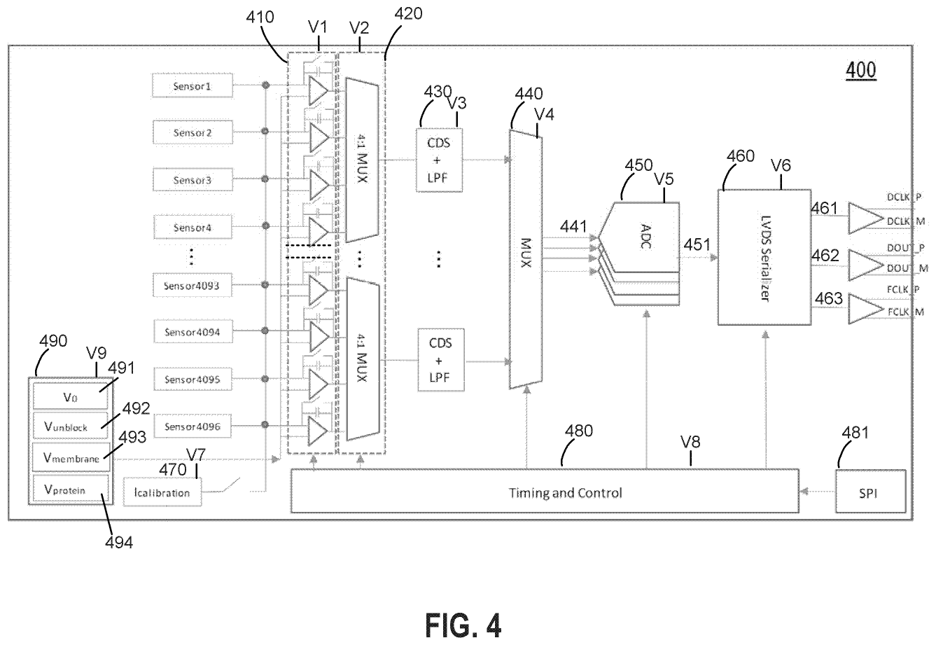

FIG. 4 is a simplified block diagram illustrating a multiple-sensor integrated chip architecture 400 for a nanopore flow cell (NFC) system according to various embodiments of the present disclosure. Referring to FIG. 4, the architecture 400 may be configured to include a plurality of amplifier clusters 410, each of the amplifier clusters 410 may include multiple amplifiers configured to amplify signals received from a plurality of sensors (sensor 1, . . . , sensor 4096). In one example, the received signals are associated with data signal associated with the sensor chip 120 through the through-silicon vias 160. The architecture 400 also includes a plurality of analog multiplexers 420, each of the analog multiplexers 420 is coupled to an output of an amplifier for selecting any one of the plurality of amplified signals of the sensors. The selected signals may be multiplexed through a multiplexer 440 to provide the signals 441 to a set of analog-to-digital converters (ADCs) 450, which convert the signals to digital data 451 representative of the analog signals 441. It is noted that the multiplexers 440 may include multiple multiplexer stages, but only one stage of multiple multiplexer stages 440 is shown for clarity purposes. In one embodiment, the architecture 400 may also include a correlated double sampling (CDS) and low-pass filtering (LPF) circuit 430 disposed between the analog multiplexers (4:1 MUX) 420 and the multiplexers (MUX) 440. The correlated double sampling and low-pass filtering (CDS+LPF) circuit 430 is configured to remove noise during reset operations of the sensors and the offset voltage of the amplifiers. The architecture 400 further includes low voltage differential signal (LVDS) interfaces 460 for transmitting differential data clock signals 461, differential data signals 462, and differential frame signals 463 to an external field programmable gate array (FPGA). The architecture 400 also includes a build-in self calibration and test (Icalibration) circuit 470 configured to provide reference signals to the amplifiers for calibration, and a timing and control signal generator 480 that generates timing clocks and control signals for driving the amplifiers 410, the multiplexers 420 and 440, the CDS and LPF circuit (CDS+LPF) 430, the ADCs 450, and the LVDS interfaces 460. The architecture 400 also includes a serial peripheral interface (SPI) port 481 configured to receive control data and/or software commands provided by a user or an external device, such as a PC or an FPGA. One skilled in the art will appreciate that other bus ports may also be used, such as an I2C port, a parallel port, a general purpose input and output (GPIO) port, a universal serial bus (USB), a short-range wireless port, a WiFi port, or the like. The architecture 400 further includes an advanced functions block 490, such as functions for applying a reference voltage V0 491 to the amplifiers, unblocking DNA strands 492 in nanopores, for nanopore membrane characterization 493, channel shorting prevention, protein insertion 494, etc. In some embodiments, all of the above enumerated blocks, devices and/or circuits 410 to 490 of the architecture 400 are integrated into an integrated chip, e.g., a CMOS integrated circuit.

In some embodiments, the architecture 400 also includes providing different voltage supplies to the different functional blocks. The operations of the signal amplification, the multiplexing, the analog-to-digital conversion, the timing control, the high-speed interface can be disruptive to the voltage supplies, thereby affecting the performance of the integrated circuit. For example, amplifiers, multiplexers, analog-to-digital converters powered by a noisy voltage supply will have noise signal readouts. Some embodiments provide different voltage supplies to the different functional blocks. For example, the amplifier clusters are provided with a voltage supply V1, the 4:1 multiplexers are provided with a voltage supply V2, the CDS and low-pass filter circuit 430 is provided with a voltage supply V3, the one or more stages multiplexers 440 are provided with a voltage supply V4, the ADCs 450 are provided with a voltage supply V5, the LVDS serializer interface 460 is provided with a voltage supply V6, the calibration and built-in self-test circuit 470 is provided with a voltage supply V7, the timing and control signal generator (or circuit) 480 is provided with a voltage supply V8, and the advanced functions block 490 is provided with a voltage supply V9. The voltage supplies V1 to V9 may have the same nominal voltage or different nominal voltages, and are physically and electrically separated from each other (e.g., through one or more dielectric layers). In one embodiment, some of the voltage supplies V1 to V9 may also be shared by other components (not shown), such as phase-locked loops (PLLs), other inputs and outputs ports to the FPGA, etc. In one embodiment, the voltage supplies V1 to V9 may be low-dropout voltage (LDO) regulators. In one embodiment, the LDO regulators are integrated with the integrated circuit (CMOS ASIC).

In one embodiment, the amplifiers in the amplifier clusters 410 are differential amplifiers having a first input coupled to sensor signals and a second input coupled to a reference voltage provided by an advanced function block 490 having various advanced functions, such as a reference voltage V0 491, a DNA unblocking function 492, a membrane characterization function 493, a protein insertion function 494, and others. In some embodiments, advanced function block 490 may also include other functions, such as some house-keeping functions. Examples of some house-keeping functions may be short circuit checking and prevention function, junction temperature verification function, channel shut-off function, self-test and/or user initiated test. Of course, one skilled in the art will appreciate that many other additional functions are possible.

The inventors observed that the likelihood of the sensors captured single biological material, such as nanopore protein and motor protein etc., in the sensor chip of the flow cell in a given time duration is about 33 percent, i.e., the occupancy of the sensors is about one third based on the Poisson distribution model. That is, experimental results show that about one third of the total sensors provide meaningful data. The inventors concluded that, by readout the sensors in a group of four, a reasonable amount of data could be collected from the flow cell and suggested to readout the sensors in a group of four. Accordingly, in accordance with the present disclosure, each of the amplifier clusters includes four differential amplifiers. It is understood that the number of sensors can be any integer number. In the example shown in FIG. 4, 4096 sensors are used. But it is understood that the number is arbitrary chosen for describing the example embodiment and should not be limiting.

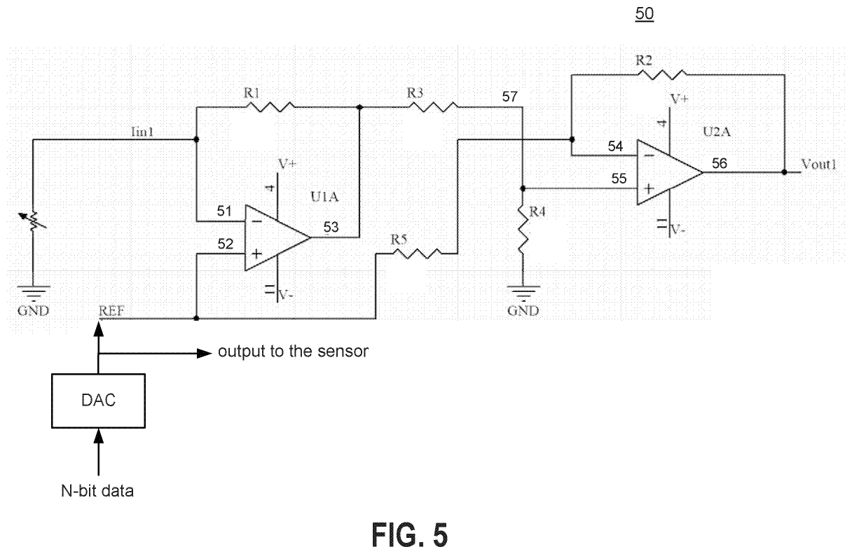

FIG. 5 is a schematic block diagram illustrating a two-stage amplifier 50 according an embodiment of the present disclosure. As shown in FIG. 5, the two-stage amplifier 50 includes a first differential amplifier or operational amplifier U1A having a first input 51 configured to receive a data signal Iin1 of a sensor, a second input 52 for receiving a reference signal REF, and an output 53 coupled back to the first input 51 through a first resistor R1. The two-stage amplifier 50 also includes a second differential amplifier or operational amplifier U2A having a first input 54 configured to receive the reference signal REF through a resistor R5, a second input 55 for receiving an amplified signal 57 from differential amplifier U1A through a resistor R3, and an output 56 coupled back to first input 54 through a resistor R2. The second input 55 of the second differential amplifier U2A is coupled to ground through a resistor R4. It is noted that the resistors are shown as discrete resistors, one skilled in the art will appreciate that other resistor configurations can also be used without limiting the scope of the present disclosure. For example, each of the resistors may include a plurality of resistors switched in series and/or in parallel, or the resistors are implemented as MOS field-effect transistors configured as variable resistors. In one embodiment, the two-stage amplifier 50 may be implemented as a transimpedance amplifier (TIA) with a feedback resistor that is adjustable to maintain the gain of the amplifier. The two-stage amplifier 50 has a two-stage amplification with the first stage amplification (gain) greater than the amplification of the second stage.

In one embodiment, the reference voltage REF is generated by an on-chip digital-to-analog converter (DAC) which converts N-bit data received from an external device (e.g., the FPGA) to an analog signal, where N is a positive integer. In one embodiment, the reference voltage REF is provided to the sensors (nanopores) of the sensor chip through an output.

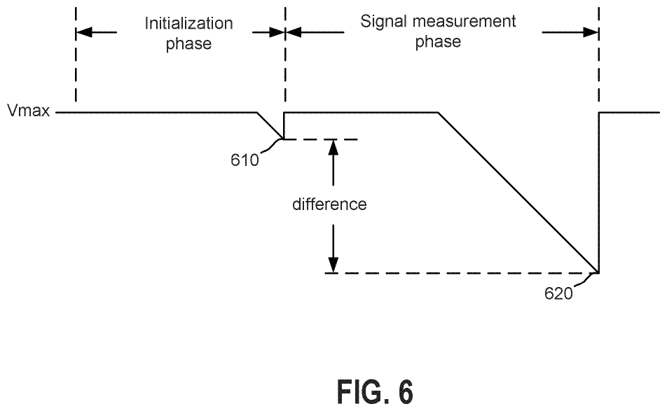

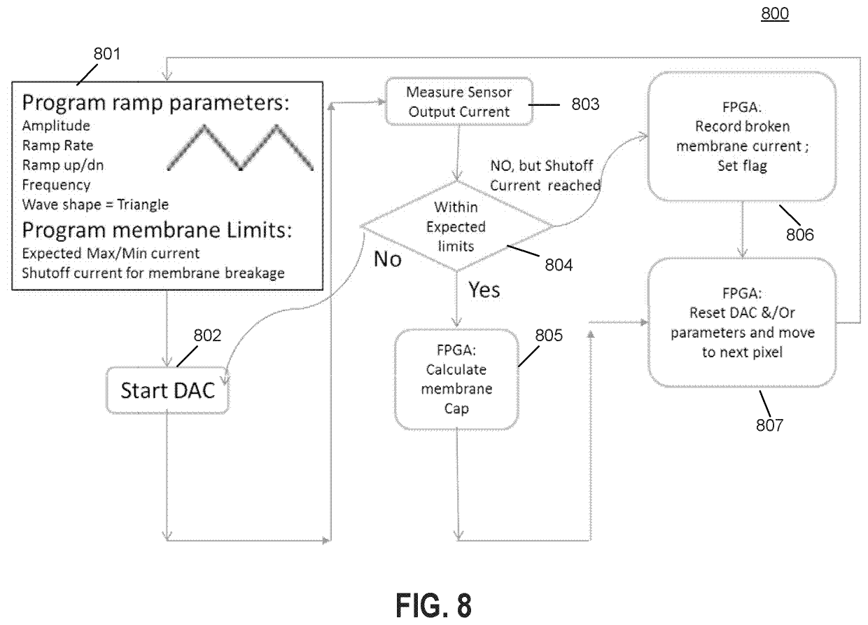

FIG. 6 is a timing diagram illustrating an exemplary readout cycle of a nanopore based on a correlated double sampling (CDS) process according to an embodiment of the present disclosure. Referring to FIG. 8, during the initiation phase where the nanopore of the flow cell has not been loaded with a biological or biochemical material, the sensor element associated with the nanopore outputs a maximum voltage Vmax. Alternatively, the unloaded nanopore of the flow cell may be precharged with the maximum voltage by the ASIC. This maximum voltage is sampled by the CDS of the CDS+LPF circuit 430 to obtain a first sampling result 610. The first sampling result 610 represents noise and offset voltage of the sensor element, the amplifier, and the multiplexer. Thereafter, the nanopore is then loaded with the biological material, and an electrical signal of the nanopore of the sensor chip is then provided to the integrated circuit (ASIC) for signal measurement. The CDS+LPF circuit 430 samples the electrical signal to obtain a second sampling result 620. The difference between the first and second sampling results represents the effective electrical signal value of the loaded nanopore without the noise and offset values. In one embodiment, the sensor chip includes a CMOS image sensor. In other words, the pixels of the CMOS image sensor are the sensor elements. The pixels can be reset to a reset voltage and converted to pixel signals during the reset phase when the flow cell is clean, i.e., the flow cell has not been loaded with a biological material. The initial electrical signals of the pixels are sampled by the CDS+LPF circuit 430 to obtain a plurality of first sampling results. Thereafter, the biological material is loaded into the flow cell through the inlet, the biological material is then absorbed into the nanopores and causes a change in the electrical signals of the pixels in the CMOS image sensor. The electrical signals of the pixels are amplified or scaled by the amplifiers, selectively passed through the multiplexers, and then sampled by the correlated double sampling circuit. In one embodiment, the effective voltage values of the pixels are the differences between the first and second sampling values. The effective voltage values are then low-pass filtered by the CDS+LPF circuit 430 to reduced uncorrelated noise. In some embodiments, the correlated double sampling is performed after a nanopore is loaded.

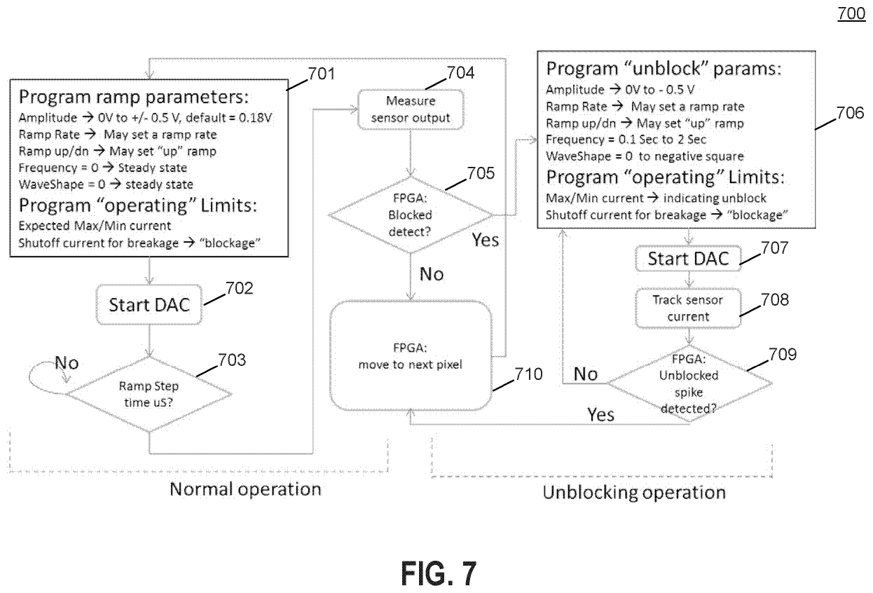

FIG. 7 is a simplified flow chart illustrating a DNA unblocking process 700 according to an embodiment of the present disclosure. Referring to FIG. 7, the DNA unblocking process includes a normal operation and an unblocking operation. In the normal operation, an DNA insertion is initiated, and a DNA current is calibrated. This will use the same algorithm for normal sequencing, except inputs may be from the built-in-self test (BIST) block or from a user indicated known DNA. At block 701, a set of operation parameters are entered and stored. At block 702, user provided data or pre-programmable data are provided to the analog-to-digital converter (DAC) which provides a corresponding voltage reference to the sensor chip via the outputs of the ASIC. At block 703, the voltage ramps are generated and checked. The electrical signals of the sensors are then measured by the DACs and sent the measured data to the FPGA at block 704. At block 705, the FPGA determines whether or not a nanopore has been blocked. When the FPGA determines that the nanopore has been blocked (yes at block 705), the DNA unblocking process 700 proceeds to program the ASIC with a set of unblock parameters (block 706) and initiates the unblocking by applying a new voltage reference to the nanopore (block 707) and tracking the electrical signal of the sensor (nanopore) at block 708. At block 709, the FPGA determines that the unblocked spike has been detected (yes in block 709) and repeats the operations to the next nanopore (sensor) at block 710. When the FPGA determines that the unblocked spike has not been detected (no in block 709), the DNA unblocking process 700 goes back to block 706 and repeats the processes until the FPGA detects the unblocked spike.

FIG. 8 is a simplified flow chart illustrating a membrane characterization process 800 according to an embodiment of the present disclosure. The membrane characterization process 800 initiates a membrane formation at block 801 by entering a set of parameters to the ASIC. In one embodiment, the ramp Vref may be from 0 V to ±500 mV for a time duration in a range between 1 ms to 10 seconds. At block 802, a DAC outputs the voltage reference to the sensor. An electrical signal of the sensor is read out and then measured at block 803. At block 804, the membrane characterization process 800 determines whether or not the electrical signal is within expected limits (or a predetermined range). When the membrane characterization process 800 determines that the electrical signal is within the expected limits or the predetermined range (yes at block 804), the FPGA proceeds to calculate the membrane capacitance value and proceeds to the next sensor (pixel). In one embodiment, the membrane capacitance value is in a range between 1 pF and 400 pF. In the event that the electrical signal is not within the expected limits but reaches a shutoff current, the FPGA determines the membrane is broken and sets a flag indicating that broken membrane. In the event that the electrical signal is not within the expected limits and does not reach the shutoff current, the process 800 proceeds back to block 802.

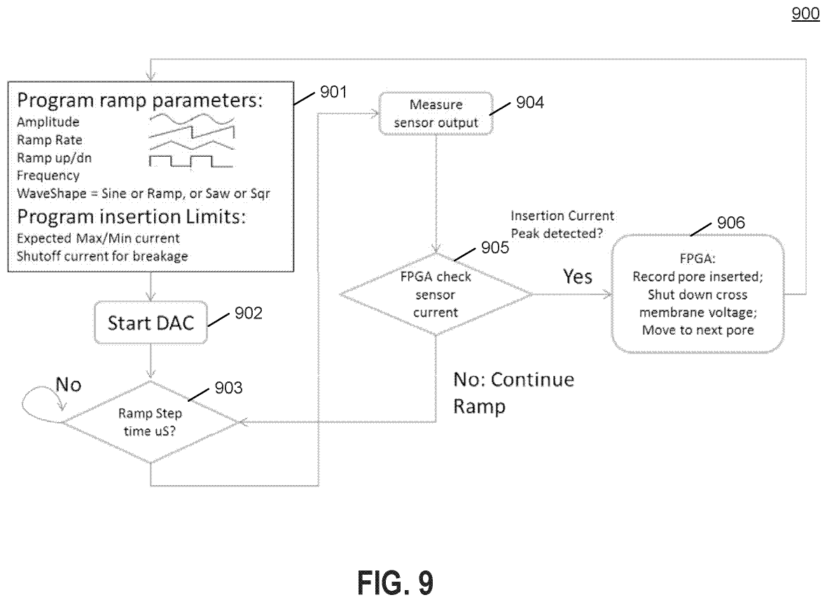

FIG. 9 is a simplified flow chart illustrating a protein insertion process 900 according to an embodiment of the present disclosure. The nanopore insertion process 900 is initiated at block 901 by entering a set of parameters to the ASIC. In one embodiment, the voltage reference Vref may be from 0 V to ±500 mV for a time duration in a range between 1 ms to 10 seconds. At block 902, a DAC outputs the voltage reference to the sensor. At block 903, the DAC continues outputting a ramp voltage. An electrical signal of the sensor is read out and then measured at block 904. At block 905, the FPGA determines the presence of a peak insertion current (Yes at block 905), the FPGA records that the nanopore has been successfully inserted to the membrane electrical signal, shuts down the cross membrane voltage and moves to the next nanopore. If the presence of the peak insertion current is not detected (No at block 905), the protein insertion process continues the voltage ramps at block 903.

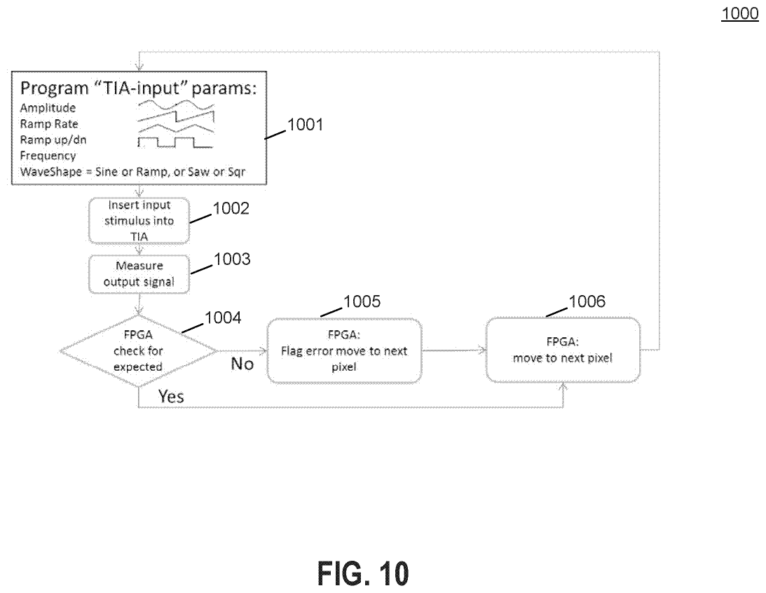

FIG. 10 is a simplified flow chart illustrating a short-circuit prevention process 1000 according to an embodiment of the present disclosure. The short-circuit prevention process 1000 is used to protect or prevent the system from short circuit conditions by performing constantly checks for short-circuit current. The short-circuit prevention process 1000 will shut off the short channels by providing registers for setting 0V bias voltage across a pixel to shut off the pixel.

Referring to FIG. 10, the short-circuit prevention process 1000 is initiated at block 1001 by entering a set of parameters to the ASIC. At block 1001, a generator for generating a set of electrical signal patterns to the amplifiers (TIA) is provided. The electrical signal waveforms can be sine waves, linear or non-linear ramps, saw patterns, square waves, or other waveforms. An electrical signal pattern is applied as stimulus to a sensor (sensor element or pixel) associated with an amplifier (block 1002) and an output signal of the sensor is scaled (amplified) by the amplifier, and provided to an ADC to obtain a measured value (block 1003). The measured value is sent to the FPGA. The FPGA determines whether the measured value satisfies or meets an expected value (block 1004). When the FPGA determines that the measured value satisfies the expected value, the FPGA instructs the ASIC to apply the stimulus to a next sensor (block 1006) and repeats the process. When the FPGA determines that the measured value does not meet the expected value, the FPGA flags an error (block 1005) and moves to block 1006.

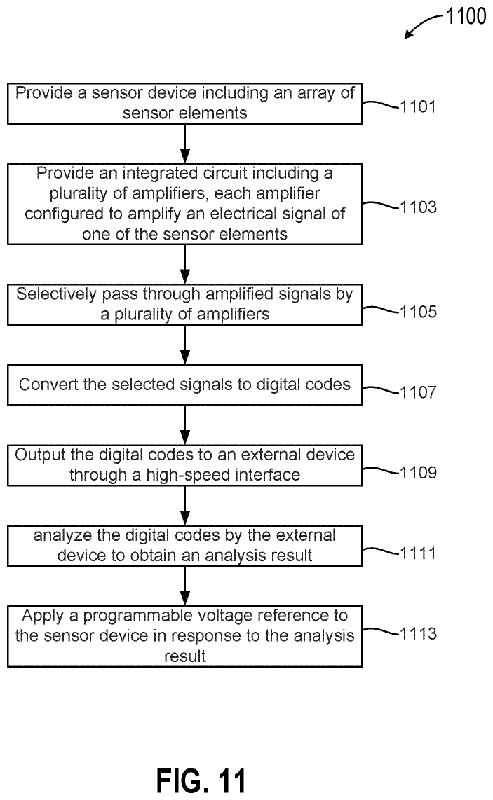

FIG. 11 is a simplified flow chart illustrating a method 1100 of operating a flow cell analysis system according to an embodiment of the present disclosure. Referring to FIG. 11, at block 1101, a sensor device is provided. The sensor device may include an array of sensor elements, each of the sensor element is configured to output an electrical signal in response to a physical condition. For example, the sensor element may be associated with a nanopore having a biological sample and outputs an electrical signal in response to the biological sample. In one embodiment, the sensor device may include a CMOS image sensor, and the sensor elements are the pixels. At block 1103, the method 1100 includes providing an integrated circuit coupled to the sensor device, the integrated circuit includes a plurality of amplifier clusters coupled to the sensor elements and configured to amplify or scale the electrical signals by a gain factor greater than unity. At block 1105, the method 1100 also includes selectively passing through some amplified electrical signals that equal to or greater than a predetermined value. The selected electrical signals are provided to a plurality of analog-to-digital converters (ADCs) through a plurality of multiplexer stages. The selected electrical signals are then digitally converted to respective digital codes representative of the selected electrical signals. The digital codes are then outputted to an external device (e.g., FPGA 13 shown in FIG. 1A) through a low voltage differential signal (LVDS) interface. In one embodiment, prior to providing the selected electrical signals to the ADCs, the method 1100 includes submitting the selected electrical signals to a correlated double sampling and filtering operation to reduce noise and offset of the sensor elements and the amplifiers. In some embodiments, a timing and control signal generator autonomously performs the amplifying, selecting, correlated double sampling and filtering, signal conversion, data storage operations. In other embodiments, these operations may be performed by a user or by a software program stored in a PC through a wire or wireless communication link (SPI, USB, WiFI, etc.).

Referring still to FIG. 11, the method 1100 further includes analyzing the digital codes by the external device at block 1111. In one embodiment, the external device analyzes the digital codes to obtain the genetic information. In another embodiment, the external device analyzes the digital codes to obtain an analysis result and determines a corrective action to the sensor device in response to the analysis result. The analysis and the corrective action may include some functions such as DNA unblocking, membrane characterization, protein insertion, short-circuit protection as described in sections above. Based on the analysis result, the external device may take corrective actions such as varying a programmable voltage reference to the sensor device through the integrated circuit (e.g., the CMOS ASIC chip) at block 1113.

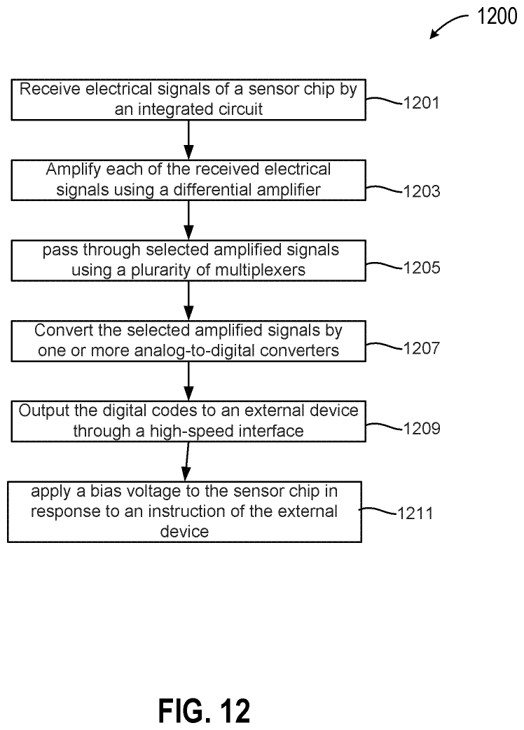

FIG. 12 is a simplified flow chart illustrating a method 1200 of operating an integrated circuit configured to control a sensor chip having a plurality of sensor elements based on an instruction received from an external device according to an embodiment of the present disclosure. Referring to FIG. 12, the method 1200 includes receiving electrical signals provided by the sensor elements by the integrated circuit at block 1201, the integrated circuit may be the CMOS ASIC device of FIG. 4 and includes a plurality of differential amplifiers, each of the differential amplifiers has a first input configured to receive an electrical signal of an sensor element and a second input coupled to a bias voltage source (programmable voltage reference) and configured to apply the bias voltage to a corresponding sensor element. At block 1203, the method 1400 includes amplifying the received electrical signals by a programmable or variable scaling factor. At block 1205, the method 1200 also includes selectively passing through the scaled (amplified) signals using a plurality of multiplexers to a number of signal paths, where the number of signal paths is smaller than the number of differential amplifiers. The selected scaled signals are then converted to digital codes by one or more analog-to-digital converters and stored at a data storage at block 1207. At block 1209, the method 1200 includes reading out or outputting the digital codes to an external device through a high-speed interface. In one embodiment, the high-speed interface includes a low voltage differential signaling (LVDS) driver for driving differential signals through a load. At block 1211, the method 1200 further includes adjusting a bias voltage and applying the bias voltage to the sensor chip.

The embodiments disclosed herein are not to be limited in scope by the specific embodiments described herein. Various modifications of the embodiments of the present invention, in addition to those described herein, will be apparent to those of ordinary skill in the art from the foregoing description and accompanying drawings. Further, although some of the embodiments of the present invention have been described in the context of a particular implementation in a particular environment for a particular purpose, those of ordinary skill in the art will recognize that its usefulness is not limited thereto and that the embodiments of the present invention can be beneficially implemented in any number of environments for any number of purposes.

1. An integrated circuit for controlling a sensor chip capable of sensing various materials, the integrated circuit comprising:
a plurality of amplifier clusters, each of the amplifier clusters comprising a number of amplifiers, each amplifier having a first input coupled to a sensor of the sensor chip, a second input coupled to a programmable voltage reference, and an output;
a plurality of first analog multiplexers, each one of the first analog multiplexers being coupled to one of the amplifier clusters and configured to selectively pass through an electrical signal; and
at least one analog-to-digital converter (ADC) coupled the analog multiplexers and configured to generate digital code values representative of electrical signals.

2. The integrated circuit of claim 1, wherein each amplifier of an amplifier cluster comprises a two-stage amplification.

3. (canceled)

4. The integrated circuit of claim 1, further comprising a digital-to-analog converter configured to generate the programmable voltage reference in response to a digital input signal, wherein the programmable voltage reference is configured to provide a bias voltage to the sensor chip for one of DNA strain unblocking, nanopore membrane characterization, or protein insertion.

5. The integrated circuit of claim 1, wherein the number of amplifiers in an amplifier cluster is four, and each first analog multiplexer comprises four inputs, each input being coupled to an output of an amplifier in the amplifier cluster.

6. The integrated circuit of claim 1, further comprising a correlated double sampling and low-pass filtering circuit coupled to the plurality of first analog multiplexers and configured to reduce noise and offset voltage and drift of the integrated circuit, and a timing and control circuit configured to provides control signals to the plurality of amplifier clusters, the plurality of first analog multiplexers, and the at least one ADC.

7. (canceled)

8. The integrated circuit of claim 1, wherein the plurality of amplifier clusters, the plurality of first analog multiplexers, and the at least one ADC are supplied by individual voltage supplies that are physically and electrically separated from each other.

9. The integrated circuit of claim 1, further comprising a self-calibration and test circuit configured to calibrate the plurality of amplifier clusters and analyze a plurality of data flows from the amplifier clusters to the at least one ADC.

10. The integrated circuit of claim 1, further comprising a plurality of second analog multiplexers arranged between the plurality of first analog multiplexers and the at least one ADC and configured to sequentially provide selectively pass through electrical signals to the at least one ADC.

11. A nanopore flow cell system comprising:
a sensor chip comprising a plurality of sensors, each sensor including a nanopore flow cell;
an integrated circuit (IC) configured to receive an electrical signal of a sensor of the sensor chip and output a digital code value representative of the electrical signal, wherein the electrical signal is configured to indicate a state of the nanopore flow cell, including:
a first state indicating a molecule passing through an unblocked state;
a second state indicating the nanopore being blocked by a molecule; and
a third state indicating the nanopore being blocked;
an interface device coupled to the integrated circuit and configured to process the digital code value received from the IC and provide control signals to the IC according to the processed digital code value,
wherein the IC comprises:
a programmable voltage reference configured to provide bias voltages for operations of the sensor chip, including at least one of nanopore membrane characterization, DNA strand unblocking, or protein insertion;
a plurality of amplifier clusters, each of the amplifier clusters comprising a number of amplifiers, each amplifier having a first input coupled to a sensor of the sensor chip and a second input coupled to the programmable voltage reference;
a plurality of analog multiplexers, each one of the analog multiplexers being coupled to one of the amplifier clusters and configured to selectively pass through an electrical signal; and
at least one analog-to-digital converter (ADC) coupled the analog multiplexers and configured to generate digital code values representative of electrical signals.

12. The nanopore flow cell system of claim 11, wherein the integrated circuit is a complementary metal oxide semiconductor (CMOS) application specific integrated circuit (ASIC), and the interface device is a field programmable gate array (FPGA).

13. The nanopore flow cell system of claim 11, further comprising a substrate disposed between the integrated circuit and the sensor chip, wherein the integrated circuit is in communication with the sensor chip through a plurality of through-silicon vias extending to the substrate.

14. The nanopore flow cell system of claim 11, wherein the integrated circuit is in communication with the interface device via a four-wire serial peripheral interface (SPI) and a low-voltage differential signaling (LVDS) port.

15. (canceled)

16. The nanopore flow cell system of claim 11, wherein the number of amplifiers in an amplifier cluster is four, and each first analog multiplexer comprises four inputs, each input being coupled to an output of an amplifier in the amplifier cluster.

17. The nanopore flow cell system of claim 11, wherein the interface device is configured to perform arithmetic operations on the digital code values received from the IC and transmits control signals to the IC according to results of the arithmetic operations.

18. The nanopore flow cell system of claim 11, wherein the sensor chip is disposed on a sensor substrate, the integrated circuit is disposed on a second substrate, the sensor chip and the integrated circuit are coupled together through a set of pogo pins.

19. A method of operating a nanopore flow cell analysis system, the method comprising:
providing a sensor chip, including an array of sensors;
providing a bias voltage to the sensor chip selected from a programmable voltage reference configured to provide bias voltage for a plurality of operations including at least nanopore membrane characterization, DNA strand unblocking, or protein insertion;
receiving an electrical signal by the IC from the sensor chip at an integrated circuit that includes a plurality of amplifiers, each amplifier configured to scale an electrical signal of one of the sensors, wherein the electrical signal is configured to indicate a state of a nanopore associated with the sensor, including a blocked state, an unblocked state, and a state of a molecule passing through an unblocked nanopore;
selectively passing through a portion of the scaled electrical signals;
converting the portion of the scaled electrical signals to digital codes representative of the portion of the scaled electrical signals;
outputting the digital codes to an external device using an interface device;
analyzing the digital codes by the external device to obtain an analysis result; and
applying a programmable voltage reference to the sensor chip in response to the analysis result.

20. The method of claim 19, further comprising multiplexing the amplified electrical signal through multiple stages of analog multiplexers prior to converting the amplified electrical signal to the digital code value.

21. (canceled)

22. The method of claim 21, wherein performing the DNA strand unblocking comprises:
detecting a blocking condition of a sensor of the sensor chip; and
changing the programmable voltage reference to a reverse bias voltage to unblock the sensor when the blocking condition is detected.

23. The method of claim 21, wherein performing the nanopore membrane characterization comprises:
determining by the interface device that the digital code value of a sensor of the sensor chip is within a predetermined range;
when the digital code value is not within the predetermined range, determining that a membrane containing the sensor is broken.

24. The method of claim 21, wherein performing the protein insertion comprises:
determining by the interface chip that the digital code value of a sensor of the sensor chip exceeds a predetermined value;
when the digital code value does not equal to or exceed the predetermined value, continue increasing the programmable voltage reference until the digital code value is equal to or exceed the predetermined value.

25. (canceled)
